(19) United States
(12) Patent Application Publication
(10) Pub. No.: US 2023/0337597 A1
(43) Pub. Date: Oct. 26, 2023

(54) MANUFACTURING METHOD FOR COMPOSITION MYCELIA, COMPOSITION MYCELIA, MANUFACTURING METHOD FOR MATERIAL COMPRISING BETA-GLUCAN

(71) Applicant: Jinhu PARK, Seoul (KR)

(72) Inventors: Jinhu PARK, Seoul (KR); Seung-Hee MOON, Seoul (KR)

(21) Appl. No.: 17/949,217

(22) Filed: Sep. 21, 2022

Related U.S. Application Data
(63) Continuation of application No. PCT/KR2022/007820, filed on Jun. 2, 2022.

(30) Foreign Application Priority Data
Apr. 20, 2022 (KR) 10-2022-0049155

(51) Int. Cl. A01G 18/00; A23F 5/02; A23L 31/00

(52) U.S. Cl. CPC A01G 18/00; A23F 5/02; A23L 31/00; A23V 2002/00

(57) ABSTRACT

The present specification provides a method of preparing a mycelium complex, which includes inputting a β-glucan-containing mushroom raw material into a medium, inputting a physiologically active material-containing plant raw material into the medium, and culturing the β-glucan-containing mushroom raw material with the physiologically active material-containing plant raw material, a mycelium complex, and a method of preparing a β-glucan-containing product.

### CROSS-REFERENCE TO RELATED APPLICATION

This application is a US Bypass Continuation Application of International Application No. PCT/KR2022/007820, filed on Jun. 02, 2022, and designating the United States, the International Application claiming a priority date of Apr. 20, 2022, based on prior Korean Application No. 10-2022-0049155, filed on Apr. 20, 2022, the disclosure of which is incorporated herein by reference in its entirety.

### BACKGROUND

### Technical Field

The present specification relates to a method of preparing a mycelium complex, a mycelium complex, and a method of preparing a beta-glucan (β-glucan)-containing product.

### Background Art

Mushrooms belonging to the division Basidiomycota are important food resources and medicinal plants that grow by decomposing various types of organic substances. Mushrooms have a unique taste and flavor depending on a type and contain a lot of proteins, vitamins, and other inorganic nutrients. According to recent studies, anticancer and antiviral effects by glucan derivatives, polysaccharides contained in mushrooms, have been reported and thus the mushrooms are receiving a worldwide spotlight as a health food. Particularly, β-glucan, a type of polysaccharide, is a material present in the cell wall of yeast, mushrooms, grains, and the like, and has the effect of inhibiting the proliferation and recurrence of cancer cells by activating the immune function of normal human cells by a non-specific immune response without directly attacking cancer cells. In addition, the immune function of immune cells, T cells and B cells, is activated by promoting the secretion of various cytokines by activating macrophages when cancer cells enter the body. In addition, β-glucan is reported to have excellent blood sugar lowering and blood cholesterol decreasing effects and improve lipid metabolism to suppress the formation and accumulation of body fat, thereby exhibiting an anti-obesity effect.

The mushrooms are commonly reported to have an excellent anti-cancer effect and immunity-strengthening effect, and are widely used in the production of pharmaceuticals as well as functional foods and health supplements.

In order to use the above-described mushrooms as a mixed raw material for pharmaceuticals and functional foods, the fruiting bodies (the mushroom caps commonly seen outdoors) of mushrooms have to be collected and used. However, there are problems in that mushrooms do not reproduce well in a natural state, and resource depletion and ecosystem destruction are caused by over-collection. In addition, a method of cultivating fruiting bodies using sawdust or the like has been used, but this method requires several months of cultivation and a large amount of production facilities and expenses to supply raw materials that can be used industrially, so it cannot meet the demand for mass production. As such, although the fruiting bodies of Inonotus obliquus, Phellinus linteus, Ganoderma lucidum, Sparassis crispa, Cordyceps Sinensis, and Hericium erinaceum are effective as anticancer agents or immune enhancers, their supply is limited and it is not easy to perform mass production and rapid production, so they are not widely used.

Recently, to contain β-glucan in cosmetics, coffee, food, or the like, technology for culturing a mycelium using it as a medium is being developed. However, there were technical limitations such as β-glucan loss or the slow growth of a mycelium. Accordingly, there is a demand for a method of preparing a mycelium complex in which a β-glucan content is maintained high.

### SUMMARY

### Technical Problem

The present specification relates to a method of preparing a mycelium complex, a mycelium complex and a method of preparing a β-glucan-containing product.

### Technical Solution

One aspect of the present invention provides a method of preparing a mycelium complex, which includes: inputting a β-glucan-containing mushroom raw material into a medium;
inputting a physiologically active material-containing plant raw material into the medium; and
culturing the β-glucan-containing mushroom raw material with the physiologically active material-containing plant raw material.

Another aspect of the present invention provides a mycelium complex prepared by the above-described preparation method.

Still another aspect of the present invention provides a method of preparing a β-glucan-containing product, which includes inoculating a product with the above-described mycelium complex.

### BRIEF DESCRIPTION OF THE DRAWINGS

FIGS. 1 and 2 are images of green coffee beans with mycelia cultured in the process of preparing coffee of Experimental Example 1-1.

FIGS. 3 and 4 are images of green coffee beans in which mycelia are partially removed in the process of preparing coffee of Experimental Example 1-1.

FIG. 5 shows the finally prepared coffee in Experimental Example 1-1.

### DETAILED DESCRIPTION

Hereinafter, the present specification will be described in detail.

In the present specification, the “β-glucan-containing mushroom raw material” refers to all types of mushrooms containing β-glucan, and its types will be described below.

In the present specification, the “beta-glucan (β-glucan)” is a type of glucan which is a type of fluorinated polysaccharide. Glucans are divided into alpha (a)-glucan and β-glucan, and α-glucans are mainly present in plant starch, and β-glucans are present in the cell walls of mushrooms, grains, and yeast. β-glucans are known to have different efficacies according to the type of extraction target. Particularly, β-glucans extracted from mushrooms are known to improve immune function and help control cholesterol and blood pressure levels.

In the present specification, the “physiologically active material-containing plant raw material” refers to a plant-derived raw material containing a physiologically active ingredient. Unless stated otherwise, its type is not limited, and the location from which the raw material is derived, for example, the stem, leaf, and root of the corresponding plant, is not limited.

In the present specification, the “physiologically active material” refers to a material serving to enhance a hormone regulatory function or immune function in the human body, and acting as an antioxidant, and its types will be described below.

Recently, as the efficacy of β-glucan contained in mushrooms has been proven, there is an attempt to include β-glucan in cosmetics or food by culturing mushrooms in coffee. However, there was a problem in that a β-glucan content was not sufficiently ensured in this process. Specifically, a method of physically mixing a mycelium with a target product to contain β-glucan has been used, and there was a limitation in that β-glucan was not sufficiently contained in the above target product due to degraded physical properties of the mycelium or a low content of β-glucan included in the mycelium.

The inventors found a method of preparing a mycelium complex that can sufficiently ensure a β-glucan content, and thus the present invention was completed. According to a method of preparing a mycelium complex to be described below, the β-glucan content of the mycelium complex was high, and when the mycelium complex was applied to a target product such as a cosmetic or food, the β-glucan content in the product was high.

In addition, by the method of preparing a mycelium complex according to one embodiment of the present invention, the mycotoxins in the prepared mycelium complex and a product to which the same is applied may be reduced, and particularly, when it is applied to coffee, the problem of elevated blood sugar that may occur in the intake of conventional coffee may be solved. For caffeine contained in coffee, liquid chromatography may be used, and a mycotoxin content may be measured by liquid chromatography and mass spectrometry. Examples of measurement conditions are as follows.
(a) Chromatography for caffeine
   Column: Scherzo SS-C18 (150 nm 4.6 mm i.d 3)
   Column Temperature: 30° C.
   Injection Volume: 5 µl
   Flow: 0.4 ml/min
   UV: 270 nm
   Solvent A: 1% acetic acid in ACN
   Solvent B: 1% acetic acid in DW
(b) Chromatography for fungi
   Column: C18 (3 mm x 150 mm, 3 µm)
   Column Temperature: 40° C.
   Injection Volume: 5 µl
   Flow: 0.5ml/min
   Solvent A: 5 mM ammonium formate solution (including 1% formic acid)
   Solvent B: 5 mM ammonium formate in methanol solution (including 0.1% formic acid)

Specifically, one embodiment of the present invention provides a method of preparing a mycelium complex, which includes: inputting a β-glucan-containing mushroom raw material into a medium; inputting a physiologically active material-containing plant raw material into the medium; and culturing the β-glucan-containing mushroom raw material with the physiologically active material-containing plant raw material. As the method of preparing a mycelium complex includes preparing a mycelium complex by culturing a β-glucan-containing mushroom raw material with a physiologically active material-containing plant raw material, the β-glucan-containing mushroom raw material is cultured with the physiologically active material, so β-glucan may not be lost during the culture. The mycelium complex prepared by the method of preparing a mycelium complex may be applied to a product requiring β-glucan, such as a cosmetic or food, and has an effect of increasing the content of β-glucan in the product.

In one embodiment of the present invention, the physiologically active material-containing plant raw material may include one or more selected from the group consisting of flavonoids, polyphenols, isoflavones, sulforaphanes, allyl compounds, limonene, and lignin. Here, the physiologically active material-containing plant raw material preferably includes a flavonoid, polyphenol, or isoflavone component.

In one embodiment of the present invention, the physiologically active material-containing plant raw material may include one or more selected from the group consisting of a black bean-derived raw material and a Ginkgo biloba-derived raw material.

In one embodiment of the present invention, the black bean-derived raw material may be Glycine max Merr. with inner color-greenish (GG), Rhynchosia volubilis Lour., and Glycine max Merr. with inner color-yellow (GY).

In one embodiment of the present invention, the Rhynchosia volubilis Lour. is called juinoonikong in Korean. The juinoonikong (Rhynchosia volubilis Lour.) has an excellent effect on blood purification and detoxification, and has been used for medicine rather than food since ancient times. In the juinoonikong (Rhynchosia volubilis Lour.), isoflavones have an excellent anticancer effect of inhibiting the growth of cancer cells, linoleic acid purifies the blood to prevent diseases such as high blood pressure, arteriosclerosis, cerebral thrombosis, etc., and saponin and choline components activate the action of the kidneys, support cell synthesis, and remove fat from the body to help improve fatty liver. In addition, the juinoonikong (Rhynchosia volubilis Lour.) is rich in vitamin E to prevent and improve melasma and freckles, and anthocyanins improve the function of collagen to make the skin elastic. In addition, the lecithin component stimulates brain activity and is good for brain health.

In one embodiment of the present invention, the Ginkgo biloba-derived raw material may be ginkgo trunk, ginkgo bark, ginkgo root, ginkgo fruit, or ginkgo leaves. In terms of collectability and the ease of processing, ginkgo leaves are preferable.

In one embodiment of the present invention, the method of the present invention includes pretreating the Ginkgo biloba-derived raw material. This step is to remove impurities included in the Ginkgo biloba-derived raw material. Particularly, when the Ginkgo biloba-derived raw material is ginkgo leaves, detoxifying the ginkgo leaves to remove cyanide compounds contained in ginkgo leaves may be included. The step of detoxifying the ginkgo leaves may be performed by immersing the ginkgo leaves in water and boiling them.

In one embodiment of the present invention, the ginkgo leaves contain 20 or more types of flavonoids. Flavonoids are considered to control the germination and growth of seeds and absorb the UV rays of the sun to protect the internal tissue, and serves to protect capillaries in the human body. Particularly, ginkgo leaves contain various antioxidant components including ginkgolide, so it is known that the ginkgo leaves eliminate blood clots and increase the elasticity of the blood vessel wall, inhibit reactive oxygen species or platelet activation factors, and have various pharmacological effects on the blood flow in the brain, the heart disease, etc.

In one embodiment of the present invention, the physiologically active material-containing plant raw material may include a black bean-derived -derived raw material and a Ginkgo biloba-derived raw material.

In one embodiment of the present invention, the black bean-derived raw material and the Ginkgo biloba-derived raw material may be added and mixed in a weight ratio of 3:7 to 7:3, and preferably 6:4 to 4:6. When the above raw materials are mixed and added as described above, it is preferable because it is possible to obtain, particularly, a mycelium complex including a high content of β-glucan.

In one embodiment of the present invention, the content of the physiologically active material-containing plant raw material may be 1 to 50 parts by weight based on 100 parts by weight of the β-glucan-containing mushroom raw material. Preferably, the content is 10 to 50 parts by weight or 20 to 40 parts by weight. When the content satisfies the above range, the amount of mushroom raw material is sufficiently ensured to stably maintain a culture rate, and the effect of increasing the β-glucan content caused by the physiologically active material may be ensured.

In one embodiment of the present invention, the physiologically active material-containing plant raw material may be prepared in a powder or liquid form. In this case, it may be easily mixed with the mushroom raw material to improve processability. For example, when the physiologically active material-containing plant raw material is juinoonikong (Rhynchosia volubilis Lour.), a method of preparing a powder form is as follows:

i) Juinoonikong (Rhynchosia volubilis Lour.) is washed 3 or 4 times, and soaked in water approximately 2 to 5 times the amount of the beans. The soaked beans are put into a pot with water 2 to 5 times the amount of the beans, boiled at high heat for 3 to 20 hours, drained, and then transferred to a fermenter. After rice straw is put into the fermenter, the lid of the fermenter is closed, and then fermentation is carried out for 2 to 7 days in a warm environment of 40 to 45 degrees. The fermented juinoonikong (Rhynchosia volubilis Lour.) is dried and ground with a grinder, thereby preparing a powder.

Meanwhile, when the physiologically active material-containing plant raw material is ginkgo leaves, the preparation method thereof is as follows:

ii) The fermented ginkgo leaf powder is prepared by washing ginkgo leaves, grinding them using a grinder, and fermenting them at room temperature for 3 to 10 days. Subsequently, the fermented product of the ground ginkgo leaves is sterilized by thermal treatment (e.g., at 100 to 120° C. for 0.5 to 2 hours). Afterward, 0.5 to 4 parts by weight of lactic acid bacteria were inoculated with respect to 100 parts by weight of the fermented product of the ground ginkgo leaves, and fermented at 35 to 38° C. for 3 to 10 days. The fermented product of the ground ginkgo leaves is dried and ground by a grinder, thereby obtaining a powder.

Here, an activation material may be added to the ground ginkgo leaves, and the content of the activation material may be 1 to 5 parts by weight based on 100 parts by weight of the ground ginkgo leaves.

As the lactic acid bacteria, lactic acid bacteria known in the art may be used without limitation, and selected from, for example, Lactobacillus acidophilus, Streptococcus thermophiles, and Bifidobacterium animalis spp. Lactis BB-12.

In one embodiment of the present invention, the physiologically active material-containing plant raw material may be fermented. In this case, the content of the physiologically active material may increase.

In one embodiment of the present invention, the β-glucan-containing mushroom raw material may include one or more selected form the group consisting of Cordyceps Sinensis, Fomes osmentarius, Ganoderma lucidum, antler-shaped Ganoderma lucidum, Sparassis crispa, Inonotus obliquus, Phellinus linteus, Lentinus edodes (Berk.) Sing, Coriolus versicolor, Pleurolus ostreatus Ganoderma, Panaeolus spp., and psilocybin mushrooms. The psilocybin mushrooms are psilocybin-containing hallucinogenic mushrooms, accounting for most types of hallucinogenic mushrooms.

Cordyceps Sinensis is known to have excellent effects on immunity enhancement, anticancer action, stress reduction, and fatigue recovery. In addition, Cordyceps Sinensis is also known to have effects on energy reinforcement, the control of a cholesterol level, blood pressure regulation, and pernicious anemia treatment.

Ganoderma lucidum grows on hardwood roots in summer. It is also known as the Qin Shi Huang’s elixir plant, and is placed in the ranks of medicinal herbs along with ginseng in Ben Cao Gang Mu (Compendium of Materia Medica). Ganoderma lucidum has tonic, antitussive and swelling-reducing effects, is effective for a respiratory disease, neurasthenia, a heart disease, and high blood pressure, and is also known to reduce cholesterol and have an anticancer effect.

Sparassis crispa grows while agglomerating on a trunk or stump near the root of a living tree from summer to autumn. Sparassis crispa looks like a bunch of white flowers and has a scent similar to pine mushroom. It grows wild at the edge of stumps cut from coniferous trees in autumn. Sparassis crispa is known to have effects of immune improvement, control of high blood pressure, suppression of blood sugar increase, improvement in blood circulation, and hematopoiesis.

Inonotus obliquus is a sclerotium that is parasitic on birch and alder trees growing wild in cold regions such as Russia, Canada, and Hokkaido in Japan. Recently, in Japan, Inonotus obliquus has been reported to be effective in preventing hepatitis C and treating liver cancer, and in the United States, Inonotus obliquus is classified as a special natural substance and being developed as future pharmaceutical and health food. In Korea, as a result of using Inonotus obliquus for gastric cancer and diabetes patients as folk remedies, it has been reported that the effect was superior to that of other mushrooms. In addition, it has been reported that Inonotus obliquus is effective in increasing body resistance, inhibiting tumor occurrence and whitening.

Phellinus linteus is also called woody mud mushroom (Mokjil-jinheuk-beoseot), and in Dongui Bogam, recorded under the name of Sang Mokyi in the section of decoction. Phellinus linteus grows wild on the trunk of a mulberry tree and is yellow in all parts except the surface of the cape. Phellinus linteus is even called Suseol because of looking like a lump of mud at first, but when grown, it looks like it is sticking its tongue out on the tree stump. Phellinus linteus has been used for uterine bleeding and menstrual irregularities since ancient times, and has recently been reported to have excellent effects in tumor suppression, immunity enhancement and whitening.

Lentinus edodes (Berk.) Sing is an edible mushroom belonging to the genus Lentinula, the family Omphalotaceae and the division Basidiomycota, and is known to improve diabetes, obesity, arteriosclerosis and high blood pressure, as well as to reduce blood pressure and promote the production of interferon, which is an antiviral substance. In addition, lentinan contained in Lentinus edodes (Berk.) Sing is known to have anticancer and antitumor actions, and since ergosterol is converted to vitamin D2 when exposed to UV rays of the sun, Lentinus edodes (Berk.) Sing is known to prevent rickets and treat anemia.

Pleurolus ostreatus Ganoderma is rich in dietary fiber and vitamins, and contains abundant dietary fiber that suppresses blood sugar elevation and discharges cholesterol from the blood. In addition, Pleurolus ostreatus Ganoderma also contains a lot of vitamin D which promotes fat breakdown, so it has the effect of purifying the blood and lowering the glycemic index. Pleurolus ostreatus Ganoderma contains β-glucans, selenium, and an RNA complex, so it has an excellent anticancer effect.

In one embodiment of the present invention, the step of preparing a mycelium complex by culturing a β-glucan-containing mushroom raw material with a physiologically active material-containing plant raw material may include performing primary culture of the β-glucan-containing mushroom raw material; and performing secondary culture by adding the physiologically active material-containing plant raw material to the primary culture product. Meanwhile, the primary culture of the β-glucan-containing mushroom raw material and the adding of the physiologically active material-containing plant raw material to the β-glucan-containing mushroom raw material may be performed at the same time.

In one embodiment of the present invention, the primary culture of the β-glucan-containing mushroom raw material may be carried out by a method of inoculating the β-glucan-containing mushroom raw material into a medium and culturing the material.

In one embodiment of the present invention, when there are two or more β-glucan-containing mushroom raw materials, the method may include preparing a mycelium by independently inoculating each mushroom raw material into a different medium and culturing the material; preparing a powder of each mycelium by grinding the mycelium; preparing a mycelium powder mixture by mixing the prepared mycelium powders; and inoculating the mycelium powder mixture into a medium and culturing the mixture.

In one embodiment of the present invention, the mycelium powder mixed in the mycelium powder mixture may be the same or different for each mushroom raw material. For example, when a mycelium powder mixture is prepared by mixing powders of Cordyceps Sinensis mycelium, Fomes osmentarius mycelium, Ganoderma lucidum mycelium, Sparassis crispa mycelium, Inonotus obliquus mycelium, Lentinus edodes (Berk.) Sing mycelium, and Pleurolus ostreatus Ganoderma mycelium, the weight ratio of the mycelium powders may be 1 to 3 : 1 to 3 : 1 to 3 : 1 to 3 : 1 to 3 : 1 to 3 : 1 to 3.

In one embodiment of the present invention, the medium used in the primary culture of the β-glucan-containing mushroom raw material may be potato dextrose agar (PDA).

In one embodiment of the present invention, the step of performing secondary culture by adding the physiologically active material-containing plant raw material to the primary culture product may be included. This step is to increase a β-glucan content by adding the physiologically active material-containing plant raw material to the primary culture product, and to obtain a nutritionally excellent mycelium by adding a physiologically active material.

In one embodiment of the present invention, the secondary culture may be performed at the time of blooming the hyphae of the β-glucan-containing mushrooms in the primary culture. Hypha is a generic term for a fungal system with a long branched fibrous structure, and the time of hypha blooming is able to be confirmed with the naked eye. As the secondary culture is performed at the time of blooming of the hypha of the β-glucan-containing mushroom in the primary culture, it is possible to prevent undesired side reactions such as fungal growth, and ensure a sufficient β-glucan content.

In one embodiment of the present invention, each of the primary culture of the β-glucan-containing mushroom raw material; and the secondary culture performed by adding the physiologically active material-containing plant raw material to the primary culture product may be performed for 5 to 14 days, at 20 to 30° C. and a relative humidity of 10% to 30%. Alternatively, the primary and secondary cultures may be performed at 27 to 28° C. and a relative humidity of 18% to 25%.

In one embodiment of the present invention, the method of preparing a mycelium complex may include isolating the mycelium complex from the medium.

One embodiment of the present invention provides a mycelium complex prepared by the above-described method of preparing a mycelium complex.

One embodiment of the present invention provides a method of producing a β-glucan-containing product, which includes inoculating a product with the above-described mycelium complex.

In one embodiment of the present invention, the product may include any one or more selected from the group consisting of cosmetics, green coffee beans, and fruits.

In one embodiment of the present invention, the product is green coffee beans, and a step of culturing the mycelium complex on green coffee beans may be included.

In one embodiment of the present invention, the product is green coffee beans, and a step of roasting the mycelium complex-cultured green coffee beans may be included.

In one embodiment of the present invention, the step of culturing the mycelium complex on green coffee beans may be performed for 5 days to 4 weeks. Alternatively, the culturing may be performed for one week to 4 weeks, 1 week to 3 weeks, or 1 week to 2 weeks.

In one embodiment of the present invention, the step of culturing the mycelium complex on green coffee beans may be performed at 15 to 30° C. and a relative humidity of 20% to 80%. Preferably, the temperature condition may be 19 to 30° C., and the relative humidity condition may be 30% to 60%. Under the duration, temperature and humidity conditions, the green coffee beans may sufficiently serve as media, make the mycelium complex grow well, and impart sufficient flavor.

In one embodiment of the present invention, the step of culturing the mycelium complex on green coffee beans may be repeatedly performed two or three times, and conditions for each step may be the same as or different from each other.

In one embodiment of the present invention, the step of culturing the mycelium complex on green coffee beans may be performed for 5 to 14 days, at 20 to 30° C. and a relative humidity of 10% to 30%. Alternatively, this step may be performed at 27 to 28° C. and a relative humidity of 18% to 25%.

In one embodiment of the present invention, the content of the mycelium complex in the culture of the mycelium complex on green coffee beans may be 0.1 to 30 parts by weight based on 100 parts by weight of the green coffee beans. Preferably, the content of the mycelium complex is 1 to 8 parts by weight or 2 to 7 parts by weight. When the above range is satisfied, the flavor of the coffee may be maintained.

In one embodiment of the present invention, after the step of culturing the mycelium complex on green coffee beans, thermally treating the beans at 50 to 150° C. for 10 minutes to 10 hours may be included. The thermal treatment is for improving the flavor of the coffee.

In one embodiment of the present invention, the step of thermal treatment may be performed by a method of applying hot air in a roasting drum.

In one embodiment of the present invention, after the step of culturing the mycelium complex on green coffee beans, post-treating the green coffee beans on which the mycelium complex has been cultured may be included. The post-treatment may be performed in one to three cycles. This step is to treat the mycelium complex cultured on the coffee beans suitable for roasting.

In one embodiment of the present invention, the step of roasting the green coffee beans on which the mycelium complex has been cultured may be performed at 150 to 250° C. Under the above temperature range, coffee that is not harmful to health may be prepared by reducing the production amount of a carcinogen, i.e., an acrylamide-based material, the flavor of coffee is improved, and damage to the coffee may be prevented. The roasting may be performed in a hot air, semi-hot air, or direct fire manner.

One embodiment of the present invention provides coffee containing β-glucan, which includes green coffee beans; and a mycelium complex in which a β-glucan-containing mushroom raw material is cultured with a physiologically active material-containing plant raw material.

In one embodiment of the present invention, the content of the mycelium complex may be 0.1 to 30 parts by weight based on 100 parts by weight of the green coffee beans. Preferably, the content of the mycelium complex is 1 to 8 parts by weight or 2 to 7 parts by weight. When the above range is satisfied, the flavor of the coffee may be maintained.

In one embodiment of the present invention, the value of the coffee containing β-glucan calculated by the following Equation 2 may be 15% or less, 13% or less, or 11% or less. When the above range is satisfied, the content of β-glucan is higher than that of caffeine in coffee.

$$\begin{matrix}\text{Content of caffeine relative to} \text{β} \text{-glucan content} = \\ \left(\text{caffeine content in coffee}\right) / \left(\text{β} \text{-glucan content in coffee}\right) ∗ 100 \left(\%\right)\end{matrix} \qquad \text{[Equation 2]}$$

Hereinafter, the present invention will be described in further detail with reference to Preparation Examples and Examples. However, the following examples are provided to explain the present invention in further detail, and the scope of the present invention is not limited by the following examples. The following examples may be appropriately modified and changed by those of ordinary skill in the art within the scope of the present invention.

### 1. Preparation Examples 1 and 2: Preparation of Physiologically Active Material-Containing Plant Raw Material

### <Preparation Example 1: Preparation of Fermented Juinoonikong (Rhynchosia volubilis Lour.) Powder>

Juinoonikong (Rhynchosia volubilis Lour.) was washed 3 to 4 times, and soaked in water approximately 3 times the amount of the beans for a day. The soaked beans were put into a pot with a sufficient amount of water, boiled on high heat for approximately 5 to 6 hours, drained, and transferred to a fermenter. After putting straws therein, the lid of the fermenter was closed and fermentation was carried out in a warm environment of 40 to 45 degrees for 2 to 3 days. The fermented juinoonikong (Rhynchosia volubilis Lour.) was naturally dried in the sun and ground in a grinder to prepare a powder with a size of 1000 mesh.

### <Preparation Example 2: Preparation of Fermented Ginkgo Leaf Powder>

Ginkgo leaves were washed and ground with a grinder, 2.5 parts by weight of Nuruk (traditional Korean fermentation starter) was added with respect to 100 parts by weight of the ground ginkgo leaves, and the resulting mixture was fermented at room temperature for 7 days. The fermented product of the ground ginkgo leaves was thermally treated at 100° C. for 30 minutes and sterilized. Afterward, 2 parts by weight of lactic acid bacteria were inoculated with respect to 100 parts by weight of the fermented product of the ground ginkgo leaves, and fermented at 37° C. for 7 days. The fermented product of the ground ginkgo leaves was dried and ground with a grinder to prepare a powder with a size of 1000 mesh.

### 2. Examples 1 to 3 and Comparative Examples 1 and 2: Preparation of Mycelium Complex

### <Example 1: Preparation of Fermented Juinoonikong (Rhynchosia volubilis Lour.) Powder-Added Mycelium Complex Powder>

Cordyceps Sinensis, Fomes osmentarius, Ganoderma lucidum, Sparassis crispa, Inonotus obliquus, Phellinus linteus, Lentinus edodes (Berk.) Sing and Pleurolus ostreatus Ganoderma were put into individual containers to prepare mushroom hyphae.

Mushroom hypha was separated from each of the containers with the 8 types of mushrooms and inoculated onto potato dextrose agar (PDA), followed by culturing the mycelium of each mushroom at a humidity of 20% and 27 to 29° C. for one week. Each mushroom mycelium cultured in the PDA was powdered and mixed in the same weight ratio, followed by co-inoculation of an Erlenmeyer flask containing potato dextrose broth (PDB) with the mycelium complex powder.

The Erlenmeyer flask containing the co-inoculated medium was put into a Bio-Oxygen Demand (BOD incubator, low-temperature incubator) to incubate the medium at 27 to 28° C. and a humidity of 20%, 40 parts by weight of the fermented juinoonikong (Rhynchosia volubilis Lour.) powder (prepared in Preparation Example 1) was added based on 100 parts by weight of the mycelium complex powder at the time of hypha blooming and subjected to stationary culture in a BOD incubator (Bio-Oxygen Demand incubator, low-temperature incubator) at 27 to 28° C. and a humidity of 20% for one week, and stirred daily for approximately one minute during the culture, thereby obtaining a mycelium complex.

### <Example 2: Preparation of Fermented Ginkgo Leaf Powder-Added Mycelium Complex Powder>

Cordyceps Sinensis, Fomes osmentarius, Ganoderma lucidum, Sparassis crispa, Inonotus obliquus, Phellinus linteus, Lentinus edodes (Berk.) Sing and Pleurolus ostreatus Ganoderma were put into individual fermenters, and water was poured to submerge the mushroom, followed by fermenting at room temperature for 1.5 years.

The hypha of each mushroom was isolated from the fermenters for the 8 types of mushrooms and inoculated onto PDA, and the mycelium of each mushroom was incubated at a humidity of 20% and 27 to 29° C. for one week. Each mushroom mycelium cultured in PDA was powdered and mixed in the same weight ratio, and an Erlenmeyer flask containing PDB was co-inoculated with the mycelium complex powder.

The co-inoculated medium contained in the Erlenmeyer flask was incubated in a BOD incubator (low-temperature incubator) at 27 to 28° C. and a humidity of 20%, 40 parts by weight of the fermented Rhynchosia volubilis Lour powder (prepared in Preparation Example 1) was added based on 100 parts by weight of the mycelium complex powder at the time of hypha blooming and subjected to stationary culture in a BOD incubator (low-temperature incubator) at 27 to 28° C. and a humidity of 20% for one week, and stirred daily for approximately one minute during the culture, thereby obtaining a mycelium complex.

### <Example 3: Preparation of Fermented Juinoonikong (Rhynchosia volubilis Lour.) Powder and Fermented Ginkgo Leaf Powder-Added Mycelium Complex Powder>

Cordyceps Sinensis, Fomes osmentarius, Ganoderma lucidum, Sparassis crispa, Inonotus obliquus, Phellinus linteus, Lentinus edodes (Berk.) Sing and Pleurolus ostreatus Ganoderma were put into individual fermenters, and water was poured to submerge the mushroom, followed by fermenting at room temperature for 1.5 years.

The hypha of each mushroom was isolated from the fermenters for the 8 types of mushrooms and inoculated onto PDA, and the mycelium of each mushroom was incubated at a humidity of 20% and 27 to 29° C. for one week. Each mushroom mycelium cultured in PDA was powdered and mixed in the same weight ratio, and an Erlenmeyer flask containing PDB was co-inoculated with the mycelium complex powder.

The co-inoculated medium contained in the Erlenmeyer flask was incubated in a BOD incubator (low-temperature incubator) at 27 to 28° C. and a humidity of 20%, 20 parts by weight of the fermented juinoonikong (Rhynchosia volubilis Lour.) powder (prepared in Preparation Example 1) and 20 parts by weight of the fermented ginkgo leaf powder (prepared in Preparation Example 2) were added based on 100 parts by weight of the mycelium complex powder at the time of hypha blooming and subjected to stationary culture in a BOD incubator (low-temperature incubator) at 27 to 28° C. and a humidity of 20% for one week, and stirred daily for approximately one minute during the culture, thereby obtaining a mycelium complex.

### <Comparative Example 1: Preparation of Mycelium Complex Not Including Physiologically Active Material-Containing Plant Raw Material>

Fruiting body tissues of the sterilized Cordyceps Sinensis, Inonotus obliquus, and Phellinus linteus were isolated and inoculated onto PDA, followed by incubating the mycelium of each mushroom at 27 to 29° C. and a humidity of 20% for one week. Each mushroom mycelium cultured in PDA was powdered and mixed in the same weight ratio, followed by co-inoculation of an Erlenmeyer flask containing PDB with the mycelium complex powder.

The co-inoculated medium contained in the Erlenmeyer flask was incubated in a BOD incubator (low-temperature incubator) at 27 to 28° C. and a humidity of 20% for one week, thereby obtaining a mycelium complex.

### < Comparative Example 2: Preparation of Mycelium Complex Not Including Physiologically Active Material-Containing Plant Raw Material>

A mycelium complex was prepared in the same manner as used in Comparative Example 1, except that sterilized Cordyceps Sinensis, Fomes osmentarius, Ganoderma lucidum, Sparassis crispa, Inonotus obliquus, Lentinus edodes (Berk.) Sing, and Pleurolus ostreatus Ganoderma were used as a mushroom raw material.

### 3. Experimental Example 1: Preparation of Β-Glucan-Containing Coffee

### <Experimental Example 1-1>

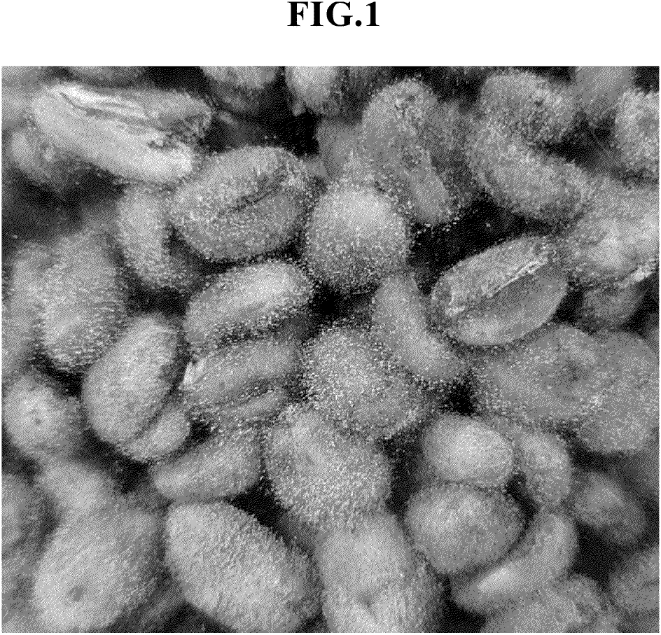

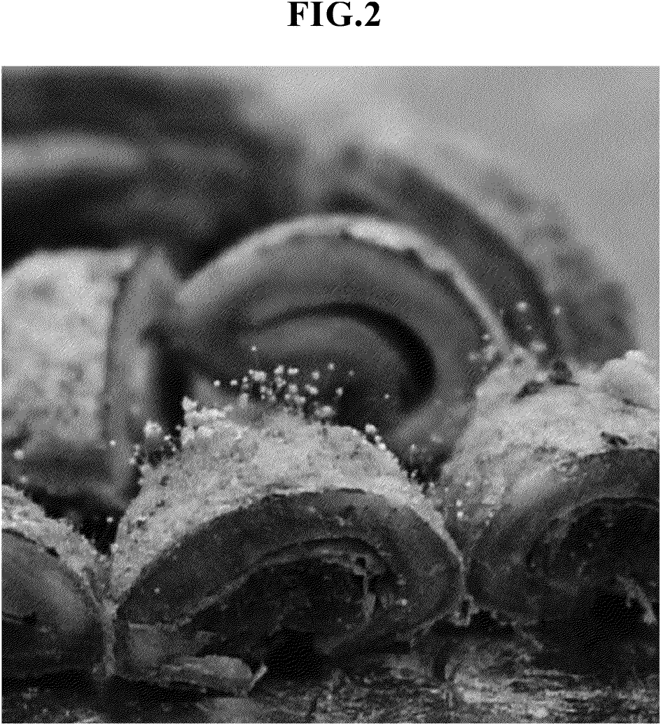

With respect to 100 parts by weight of green coffee beans, 5 parts by weight of the mycelium complex powder prepared in Example 1 was mixed and incubated at 25 to 29° C. and a relative humidity of 50%. After the hyphae bloomed by the above culture, it was further cultured at 20 to 25° C., a relative humidity of 35 to 25% for 2 weeks. Photographs of the mycelium-cultured green coffee beans are shown in FIGS. 1 and 2.

The cultured green coffee beans were put into a roasting drum, and post-treated by supplying hot air of 80 to 95° C. for one hour.

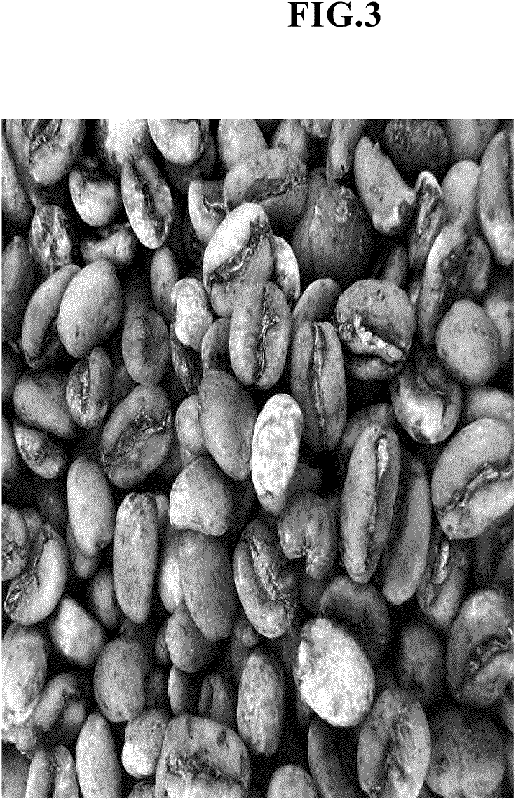

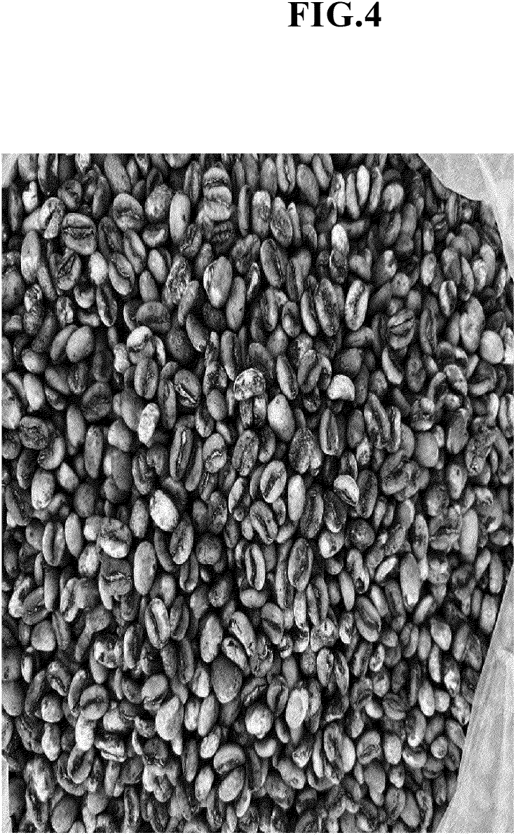

Afterward, the green coffee beans were washed with water and dried. The surfaces of the washed and dried green coffee beans are shown in FIGS. 3 and 4.

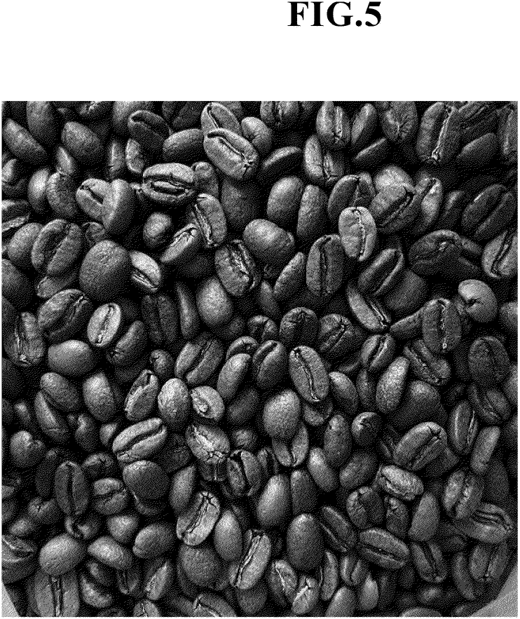

The odor-eliminated green coffee beans were directly roasted at 205° C. to prepare coffee containing β-glucan. The finally prepared coffee is shown in FIG. 5.

### <Experimental Example 1-2>

Coffee containing β-glucan was prepared in the same manner as in Experimental Example 1-1, except that the mycelium complex prepared in Example 2 was used as a mycelium complex powder.

### <Experimental Example 1-3>

Coffee containing β-glucan was prepared in the same manner as in Experimental Example 1-1, except that the mycelium complex prepared in Example 3 was used as a mycelium complex powder.

### <Experimental Example 1-4>

Coffee containing β-glucan was prepared in the same manner as in Experimental Example 1-1, except that the mycelium complex prepared in Comparative Example 1 was used as a mycelium complex powder.

### <Experimental Example 1-5>

Coffee containing β-glucan was prepared in the same manner as in Experimental Example 1-1, except that the mycelium complex prepared in Comparative Example 2 was used as a mycelium complex powder.

### 4. Experimental Example 2: Measurement of Β-Glucan Content

### <Experimental Example 2: Measurement of Β-Glucan Content>

The β-glucan content of green coffee beans before final coffee completion and the β-glucan content of the finally prepared coffee were compared.

### <Experimental Example 2-1: Measurement of Β-Glucan Content of Green Coffee Beans Before Final Coffee Completion>

2,000 mL of distilled water as an extraction solvent was added to 100 g each of the mycelium complexes obtained in Examples 1 and 2, and the mixture was extracted in a water bath at 100° C. for 24 hours and centrifuged, thereby obtaining a supernatant. In addition, after centrifugation, 2,000 mL of distilled water as an extraction solvent was added again to the resulting precipitate, and the mixture was extracted in a water bath at 100° C. for 24 hours, and repeatedly extracted a total of three times, thereby obtaining an extract. Subsequently, the extract was concentrated under reduced pressure to obtain 100 mL of a concentrate. Afterward, the temperature of the concentrate was maintained at 4° C., 300 mL of ethanol ice-cooled to -20° C. was added thereto, and the mixture was left at 4° C. for 4 hours. And then, the resulting precipitate was separated from the concentrate, and the content of β-glucan was measured using a β-glucan assay kit (Megazyme).

### <Experimental Example 2-2: Measurement of Β-Glucan Content of the Finally Prepared Coffee>

Pre-dried green coffee beans prepared in each of Experimental Examples 1-1 to 1-5 were ground, 2,000 mL of distilled water as an extraction solvent was added to 100 g of the ground green coffee beans, and the mixture was extracted in a water bath at 100° C. for 24 hours and centrifuged, thereby obtaining a supernatant. In addition, after centrifugation, 2,000 mL of distilled water as an extraction solvent was added to the resulting precipitate, and the mixture was extracted in a water bath at 100° C. for 24 hours and repeatedly extracted a total of three times, thereby obtaining an extract. The extract was concentrated under reduced pressure to obtain 100 mL of a concentrate. Subsequently, the temperature of the concentrate was maintained at 4° C., 300 mL ethanol ice-cooled to -20° C. was added thereto, and the mixture was left at 4° C. for 4 hours. Afterward, the precipitate was separated from the concentrate, and a β-glucan content was measured using a β-glucan assay kit (Megazyme).

### 5. Experimental Example 3: Measurement of Caffeine Content

The coffee containing β-glucan prepared in each of Experimental Examples 1-1 to 1-5 was ground to a size of 0.5 mm, a coffee drink sample was prepared by leaching the coffee in 5 L of purified water at 4° C. for 13 hours, and the caffeine content was measured by high performance liquid chromatography (HPLC).

### < Result Analysis>

The contents of the materials measured in Experimental Examples 2 and 3 are shown in Table 1 below.

Using the results shown in Experimental Examples 2-1 and 2-2 and Equation 1 below, the β-glucan increase amount of the coffee was calculated.

In addition, using the Equation 2 below, the content of caffeine relative to the β-glucan content was calculated.

$$\begin{matrix}\text{β} \text{-glucan increase amount} = \left\{\left(\text{β} \text{-glucan content measured in}\right)\right) \\ \left(\text{Experimental Example 2-2}\right) \text{-} \left(\text{β} \text{-glucan content measured in}\right) \\ \left(\left(\text{Experimental Example 2-1}\right)\right\} / \left(\text{β} \text{-glucan content measured in}\right) \\ \left(\text{Experimental Example 2-1}\right) ∗ 100 \left(\%\right)\end{matrix} \qquad \text{[Equation 1]}$$

$$\begin{matrix}\text{Caffeine content relative to} \text{β} \text{-glucan content} = \left(\text{caffeine content}\right) \\ \left(\text{measured in Experimental Example 3}\right) / \left(\text{β} \text{-glucan content measured}\right) \\ \left(\text{in Experimental Example 2-2}\right) ∗ 100 \left(\%\right)\end{matrix} \qquad \text{[Equation 2]}$$

TABLE 1

| Type of mycelium complex | Experimental Example 2-1 (mg/100 ml) | Experimental Example 2-2 (mg/100 ml) | Experimental Example 3 (mg/100 ml) | Equation 1 (%) | Equation 2 (%) |
|---|---|---|---|---|---|
| Example 1 | 1,430 | 2,130 | 232 | 149% | 10.9% |
| Example 2 | 1,520 | 2,765 | 219 | 182% | 7.9% |
| Example 3 | 1,612 | 3,167 | 205 | 196% | 6.5% |
| Comparative Example 1 | 5,230 | 685 | 262 | 13.1% | 38.2% |
| Comparative Example 2 | 6,878 | 1,121 | 259 | 16.3% | 23.1% |

From the above result, it can be confirmed that the coffee prepared by the preparation methods according to Examples 1 to 3 had a large content of β-glucan in the finally prepared coffee.

On the other hand, it can be confirmed that the coffee prepared by the preparation methods according to Comparative Examples 1 and 2 lost a considerably large amount of β-glucan during the preparation, and only less than 20% of the β-glucan remained compared to the initial β-glucan.

Meanwhile, it can be confirmed that, in the coffee prepared by the preparation methods according to Examples 1 to 3, the caffeine content was adjusted to less than 11% relative to the β-glucan content, and in the coffee prepared by the preparation methods according to Comparative Examples 1 and 2, the caffeine content was adjusted to more than 20% relative to the β-glucan content.

A mycelium complex prepared by a method of preparing a mycelium complex according to one embodiment of the present invention has a high β-glucan content.

The mycelium complex prepared by a method of preparing a mycelium complex according to one embodiment of the present invention has a high β-glucan content in a product when applied to the product.

What is claimed is:

1. A method of preparing a mycelium complex, comprising:
inputting a β-glucan-containing mushroom raw material into a medium;
inputting a physiologically active material-containing plant raw material into the medium; and
culturing the β-glucan-containing mushroom raw material with the physiologically active material-containing plant raw material.

2. The method of claim 1, wherein the physiologically active material-containing plant raw material comprises one or more components selected from the group consisting of flavonoids, polyphenols, isoflavones, sulforaphanes, allyl compounds, limonene, and lignin.

3. The method of claim 1, wherein the physiologically active material-containing plant raw material comprises one or more raw materials selected from the group consisting of a black bean-derived raw material and a ginkgo-derived raw material.

4. The method of claim 1, wherein the content of the physiologically active material-containing plant raw material is 1 to 50 parts by weight based on 100 parts by weight of the β-glucan-containing mushroom raw material.

5. The method of claim 1, wherein the physiologically active material-containing plant raw material is a powder or liquid form.

6. The method of claim 1, wherein the β-glucan-containing mushroom raw material comprises one or more selected form the group consisting of Cordyceps Sinensis, Fomes osmentarius, Ganoderma lucidum, antler-shaped Ganoderma lucidum, Sparassis crispa, Inonotus obliquus, Phellinus linteus, Lentinus edodes (Berk.) Sing, Coriolus versicolor, Pleurolus ostreatus Ganoderma, Panaeolus spp., and psilocybin mushrooms.

7. The method of claim 1, wherein the preparing of a mycelium complex by culturing a β-glucan-containing mushroom raw material with a physiologically active material-containing plant raw material comprises
performing primary culture of the β-glucan-containing mushroom raw material; and
performing secondary culture by adding the physiologically active material-containing plant raw material to the primary culture product.

8. The method of claim 7, wherein the secondary culture is performed at the time of blooming of the hyphae of the β-glucan-containing mushrooms in the primary culture.

9. The method of claim 1, further comprising isolating the mycelium complex from the medium.

10. A mycelium complex prepared by the method of preparing a mycelium complex according to claim 1.

11. A method of preparing a β-glucan-containing product, comprising inoculating a product with the mycelium complex of claim 10.

12. The method of claim 11, wherein the product comprises any one or more selected from the group consisting of cosmetics, green coffee beans, and fruits.
